(19) United States
(12) Patent Application Publication
(10) Pub. No.: US 2023/0340333 A1
(43) Pub. Date: Oct. 26, 2023

(54) SUSPENSION STABILIZER AGENT

(71) Applicant: SUZANO S.A., Salvador-Bahia (BR)

(72) Inventors: Estevão Frigini MAI, Vila Velha, Espirito, Santo (BR); Germano Andrade SIQUEIRA, São José dos Campos, São Paulo (BR); Matheus Antunes GUIMARÃES, Aracruz-Espirito, Santo (BR); Richieli Teles VIEIRA, Osasco-São Paulo (BR)

(73) Assignee: SUZANO S.A., Salvador-Bahia (BR)

(21) Appl. No.: 17/924,568

(22) PCT Filed: May 11, 2021
(86) PCT No.: PCT/BR2021/050197

§ 371 Date: Nov. 10, 2022

Related U.S. Application Data
(60) Provisional application No. 63/023,056, filed on May 11, 2020.

(51) Int. Cl. C09K 23/52; C08L 1/02; A61K 9/10; A61K 47/38; A61K 8/04; A61K 8/73; A61Q 19/00; A61K 8/02

(52) U.S. Cl. CPC C09K 23/52; C08L 1/02; A61K 9/10; A61K 47/38; A61K 8/044; A61K 8/731; A61Q 19/00; A61K 8/0254; C08L 2205/16; A61K 2800/10

(57) ABSTRACT

The present invention relates the use of a microfibrillated cellulose as a suspension stabilizer agent, a network structuring agent, or a network structuring additive in the manufacture of a liquid composition having a particulate system. Preferably, the microfibrillated cellulose is present from 0.5% to 5.0% in a stabilized liquid composition. The stabilized liquid composition also has a particulate system, having a plurality of particles, a plurality of capsules or mixtures thereof.

### FIELD OF INVENTION

The present invention relates the use of a microfibrillated cellulose as a suspension stabilizer agent, a network structuring agent, or a network structuring additive in the manufacture of a liquid composition having a particulate system. Accordingly, it is also provided the use of a suspension stabilizer agent in the form of a microfibrillated cellulose as a particulate system protective agent for the production of a liquid composition having a particulate system.

### BACKGROUND

WO 2009/135765 describes processes for preparing liquid compositions which comprise up to 0.2% microfibrous cellulose of bacterial origin. Even though having same chemical structure, the microfibrous cellulose produced by bacteria faces some challenges in respect to cost and scalability. On the other hand, mechanically obtained microfibrillated cellulose can be produced in large scale and with much more competitive costs.

There is still a need for the stabilization of liquid compositions having a particulate system within renewable stabilizers agents. The efficient use of MFC in the preparation of stabilized liquid compositions allows its use as a fully formulated consumer product.

### SUMMARY

The present invention relates the use of a microfibrillated cellulose as a suspension stabilizer agent, a network structuring agent, or a network structuring additive in the manufacture of a liquid composition having a particulate system. Preferably, the microfibrillated cellulose is present from 0.5% to 5.0% wt. in a stabilized liquid composition. The stabilized liquid composition also has a particulate system. The particulate system has a plurality of particles, a plurality of capsules or mixtures thereof.

Also is provided the use of a microfibrillated cellulose as a particulate system protective agent for a particulate system in a liquid composition, preferably in the range of 0.5% to 5.0% in the total weight of the stabilized liquid composition. Accordingly, it is also provided the use of a suspension stabilizer agent in the form of a microfibrillated cellulose as a particulate system protective agent for the production of a liquid composition comprising a particulate system.

### FIGURES

FIG. 1 illustrates the stabilized liquid composition MLC 0.5; MLC 1 and MLC 2, objects of the present invention, comprising respectively, 0.5%, 1% and 2% of microfibrillated cellulose and 0.5% of capsules and comparison to the liquid composition LC comprising 0.5% capsules—immediately after mixing.

FIG. 2 illustrates the stabilized liquid composition MLC 0.5; MLC 1 and MLC 2, objects of the present invention, comprising respectively, 0.5%, 1% and 2% of microfibrillated cellulose and 0.5% of capsules and comparison to the liquid composition LC comprising 0.5% capsules—after 15 days of mixing.

FIG. 3 is a microscopy of the top, middle and bottom of the liquid composition LC comprising 0.5% capsules, immediately after mixing (Initial) and 15 days after mixing (15 days).

FIG. 4 is a microscopy of the top, middle and bottom of the MLC 0.5 stabilized liquid composition comprising 0.5% capsules and 0.5% of the microfibrillated cellulose, immediately after mixing (Initial) and 15 days after mixing (15 days).

FIG. 5 is a microscopy of the top, middle and bottom of the MLC 1 stabilized liquid composition comprising 0.5% capsules and 1% of the microfibrillated cellulose, immediately after mixing (Initial) and 15 days after mixing (15 days).

FIG. 6 is a microscopy of the top, middle and bottom of the MLC 2 stabilized liquid composition comprising 0.5% capsules and 2% of the microfibrillated cellulose, immediately after mixing (Initial) and 15 days after mixing (15 days).

FIG. 7 is a graph representing the distribution of the capsule concentration in the Capsule Concentration at the top, middle and bottom of the liquid composition LC.

FIG. 8 is a graph representing the distribution of the capsule concentration in the Capsule Concentration at the top, middle and bottom of the Capsule Concentration in the MLC 0.5 stabilized liquid composition comprising 0.5% capsules and 0.5% of microfibrillated cellulose.

FIG. 9 is a representative graph of the distribution of the capsule concentration in the Capsule Concentration at the top, middle and bottom of the Capsule Concentration in the MLC 1 stabilized liquid composition comprising 0.5% capsules and 1% of microfibrillated cellulose.

FIG. 10 is a representative graph of the distribution of the capsule concentration in the Capsule Concentration at the top, middle and bottom of the Capsule Concentration in the MLC 2 stabilized liquid composition comprising 0.5% capsules and 2% of microfibrillated cellulose.

FIG. 11 is a scanning electron microscopy with detail on the LC position capsules.

FIG. 12 is a scanning electron microscopy with detail on the capsules of the MLC 0.5 comprising 0.5% capsules.

FIG. 13 illustrates the liquid composition LC comprising 0.5% capsules immediately after mixing and after 15 days.

FIG. 14 illustrates the stabilized liquid composition MLC 1 of the present invention comprising 1% of microfibrillated cellulose and 0.5% of capsules immediately after mixing and after 15 days.

FIG. 15 is a scanning electron microscopy with detail on the LC position capsules (0.5% capsules).

FIG. 16 is a scanning electron microscopy with detail on the capsules of the MLC1 with 1% of MFC comprising 0.5% capsules.

FIG. 17 shows a graph representing the number of particles counted at three different suspension levels: top, middle and bottom for the composition LC.

FIG. 18 shows a graph representing the number of particles counted at three different suspension levels: top, middle and bottom for the composition MLC1.

### DETAILED DESCRIPTION OF THE INVENTION

The present invention relates to the use of a microfibrillated cellulose as a suspension stabilizer and a network structuring agent in a liquid composition comprising a particulate system, and as a structuring additive added in the manufacture of a stabilized liquid composition comprising a particulate system, as well as a method of providing a microstructure to a liquid composition comprising a particulate system.

Microfibrillated cellulose may be produced from plant cell walls by different types of processes: simple mechanical methods, a combination of chemical and mechanical methods or enzymatic methods.

Several sources of cellulose have been used to obtain cellulose microfibers including hardwood, softwood, soybean, cotton, wheat straw, bacterial cellulose, sisal, hemp, sugar bagasse and others. Wood is the most important industrial source of cellulosic fibers from which the microfibrillated cellulose is manly extracted by mechanical methods. The disintegrated fibers are moderately degraded and opened into their sub structural fibrils and microfibrils by applying shear forces to the cellulose fiber suspension. The fibrils and their aggregates are highly entangled, inherently connected, and form mechanically strong networks and gels. The inherent interactions result in much stronger gels than those formed only by weak hydrogen bonds between water and fibrils. Additionally, to the simple mechanical methods, various pretreatments, such as enzymatic and/or chemical (oxidation, carboxymethylation, etc.) enable MFC to be obtained by a less energy consuming route.

In a first aspect, the present invention is achieved with the use of a microfibrillated cellulose as a suspension stabilizer agent in a liquid composition comprising a particulate system.

Particulate systems in liquid compositions, in general, tend to coalesce or deposit on the bottom of the compartment carrying the composition (caking), or even to float forming a supernatant. The use of the microfibrillated cellulose as a suspension stabilizing agent prevents the agglomeration of the particulate system and forms a stabilized liquid composition. The microfibrillated cellulose, when used in a liquid composition, according to the present invention, maintains the dispersion of the particulate system achieved during mixing.

In another embodiment, it is also disclosed the use of a microfibrillated cellulose to provide a network structure in a liquid composition comprising a particulate system to provide a stabilized liquid composition.

The particulate system in a liquid composition has the tendency to coalesce, aggregate, sediment or float. The use of a microfibrillated cellulose in a liquid composition provides a network structure that prevents the particulate system to coalesce, aggregate, sediment or float, forming a stabilized liquid composition.

In another embodiment, it is also disclosed the use of a microfibrillated cellulose as a suspension stabilizer agent or as a network structuring agent acting as a structuring additive added to the liquid composition in the manufacture of a stabilized liquid composition comprising a particulate system.

The use of a microfibrillated cellulose as a structuring additive that is added to the liquid composition during the manufacture of the stabilized liquid composition provides protection to the particulate system from the shear forces that are applied to the system during agitation and mixing operations.

In another embodiment, the invention is achieved with the use of microfibrillated cellulose as a suspension stabilizer agent, as a network structuring agent or as a network structuring additive in the manufacture of a liquid composition comprising a particulate system is of 0.5% to 5.0%, preferably 1.0% of a microfibrillated cellulose in the total weight of the liquid composition.

Further, the present invention also provides the use of a suspension stabilizer agent in the form of a microfibrillated cellulose to produce a stabilized liquid composition comprising a particulate system. Preferably, the use considers from 0.5% to 5.0%, preferably from 0.5% to 2.5%, more preferably 1.0% of microfibrillated cellulose during the production of a stabilized liquid composition.

It is preferred that the microfibrillated cellulose of the present invention has a diameter from 0.02 μm to 2 μm, preferably from 0.02 to 1 μm, more preferably 0.6 μm.

The stabilized liquid composition of the present invention comprises:
a liquid suspension comprising:
a microfibrillated cellulose, and
a particulate system.

The main characteristic of the elementary component of the particulate system, by elementary component it is meant the particle, capsule or microcapsule which is the target of the stabilization, is to have an interface with the surrounding environment. In general, the particulate system of interest of the present invention has some stability in the chosen liquid diluent of the liquid composition and it will not readily dissolve or solvate. Usually, when a particulate system is dispersed in a diluent it may coalesce, deposit on the bottom (caking) or form a supernatant.

A particulate system of interest to disperse in a liquid medium usually may not disperse readily, requiring the application of some mixing operation. However, once such a mixing operation ceases, the particulate system may agglomerate.

Examples of particulate systems are a plurality of particles, capsules, spheres, or their mixtures. In general, the elementary component of a particulate system is discrete and determined. It may be a single, continuous matrix, such as a particle or a matrix comprising one or more discontinuities, such as a reservoir. The elementary component of the particulate system may be of several different structures, which may be spherical, elongated, or even amorphous.

Further, the particles or capsules of the particulate system are friable, activated by moisture, activated by heat, or combinations thereof. Also, the particles or capsules of the particulate system may comprise a releasing element chosen from: a perfume, a flavoring, a softening agent, a fabric conditioning agent, a vitamin, an organic acid, a pharmaceutical active, a cosmetic active, an antistatic agent, an anti-hair loss agent, a water-repellent agent, a non-stick agent, a skin cooling agent, an antimicrobial agent, a disinfectant agent, an anti-wrinkle agent, a repellent agent, an UV protection agent, a skin conditioning agent, a skin nutrition agent, or mixtures thereof.

The form of the particles of the particulate system may be a powder, a microfiber, a microparticle, a microsphere, or mixtures thereof and the capsules of the particulate system may comprise capsules, microcapsules, or mixtures thereof.

The liquid diluent of the stabilized liquid composition may comprise water, ethanol, or a mixture.

The stabilized liquid composition can be used as a fully formulated consumer product or can be added to one or more additional ingredients to form a reformulated consumer product. Further, the stabilized liquid composition may comprise a dye, a polymer, a surfactant, a builder, a dye transfer inhibiting agent, a dispersant, an enzyme, a bleach activator, a polymeric dispersing agent, an anti-redistribution agent, a foam suppressant agent, an opacifier, and their mixtures.

If comprising a surfactant, it is selected from the group consisting of: anionic surfactant, nonionic surfactant, cationic surfactant, amphoteric surfactant and mixtures thereof.

In addition, the stabilized liquid composition can be a stabilized liquid home care composition, stabilized liquid cleaning composition, stabilized liquid tissue care composition, a stabilized liquid cosmetic composition, a stabilized liquid pharmaceutical composition.

The stabilized liquid home care composition can be a dishwashing detergent composition, an automatic dishwasher detergent, a surface cleaner, or a mixture thereof.

The stabilized liquid home care composition may comprise a surfactant, adjuvants, auxiliary cleaning agents, acid cleaning agents, metal chelating agents, calcium scavenging agents, bleaching agents, abrasives, biocidal agents, corrosion inhibitors, enzymes, and release agents.

The stabilized liquid cosmetic composition can be a shampoo, a conditioner, a pre-shampoo, a liquid soap, or any other composition suitable for cleaning or skin or hair care.

The liquid stabilized pharmaceutical composition comprises a particulate system that is a water-insoluble pharmaceutical agent.

Another embodiment of the present invention is a method of manufacturing a stabilized liquid composition, comprising the steps:
a) Providing a liquid diluent, a particulate system and a microfibrillated cellulose;
b) Mixing the liquid diluent, the particulate system and the microfibrillated cellulose, producing a microfibrillated cellulose liquid composition.

An alternative embodiment of the present invention is a method of manufacturing a stabilized liquid composition wherein the liquid diluent and the particulate system are mixed, forming a liquid particulate system mixture, followed by the addition and mixture of the microfibrillated cellulose forming a stabilized liquid composition.

An alternative embodiment of the present invention is a method of manufacturing a stabilized liquid composition wherein the liquid diluent and the microfibrillated cellulose are mixed together, forming a liquid diluent and microfibrillated cellulose mixture, followed by the addition and mixture of the particulate system forming a stabilized liquid composition. In a further alternative, the microfibrillated cellulose and the liquid diluent are mixed to provide a pre-mix for further addition to the particulate system.

An alternative embodiment of the present invention is method of manufacturing a stabilized liquid composition wherein the microfibrillated cellulose and the particulate system are mixed together forming a microfibrillated cellulose and particulate system mixture, followed by the addition and mixture of the liquid diluent forming a stabilized liquid composition.

### EXAMPLES

### Example 1

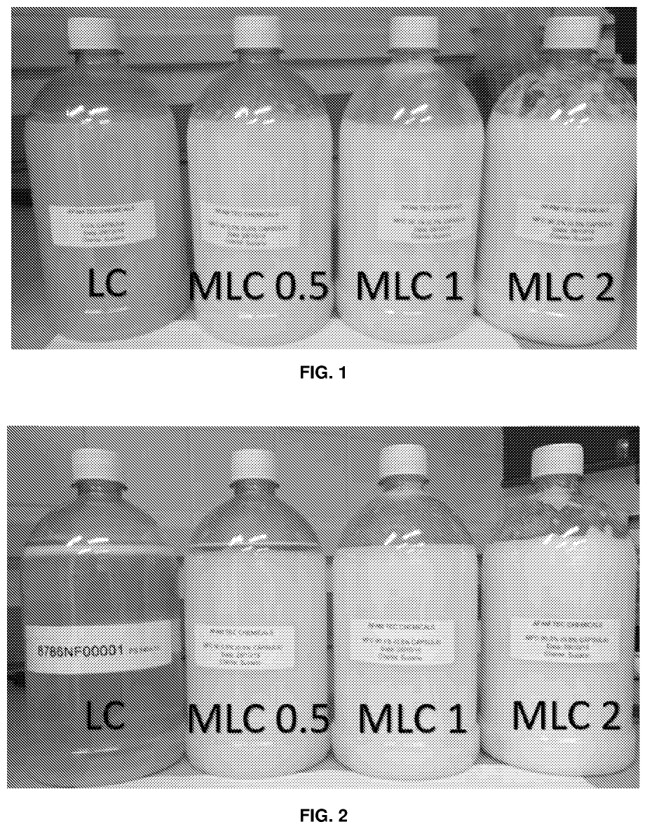

FIG. 1 illustrates a comparison between microstructured liquid compositions MLC 0.5; MLC 1 and MLC 2, objects of the present invention, comprising respectively, 0.5%, 1% and 2% wt. of microfibrillated cellulose and 0.5% of capsules in a liquid composition LC comprising 0.5% capsules and free of microfibrillated cellulose, all samples immediately after mixing.

FIG. 2 illustrates a comparison between microstructured liquid compositions MLC 0.5; MLC 1 and MLC 2, objects of the present invention, comprising respectively, 0.5%, 1% and 2% wt. of microfibrillated cellulose and 0.5% of capsules in a liquid composition LC comprising 0.5% capsules and free of microfibrillated cellulose, all samples after 15 days.

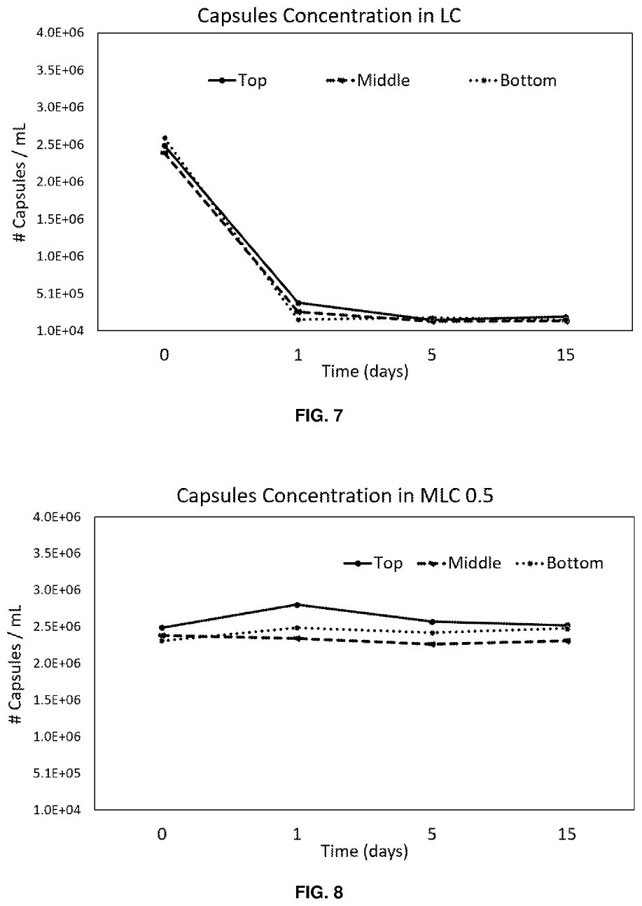

The comparison between each sample shows that the higher the amount of microfibrillated cellulose, the more stable is the dispersion provided to the liquid composition. The sample LC, after 15 days, shows the agglomeration of the particle system at the top of the container. Such agglomeration was measured and is depicted at FIG. 7. The rapid agglomeration detected at the first day after mixing could not be avoided by the particulate system.

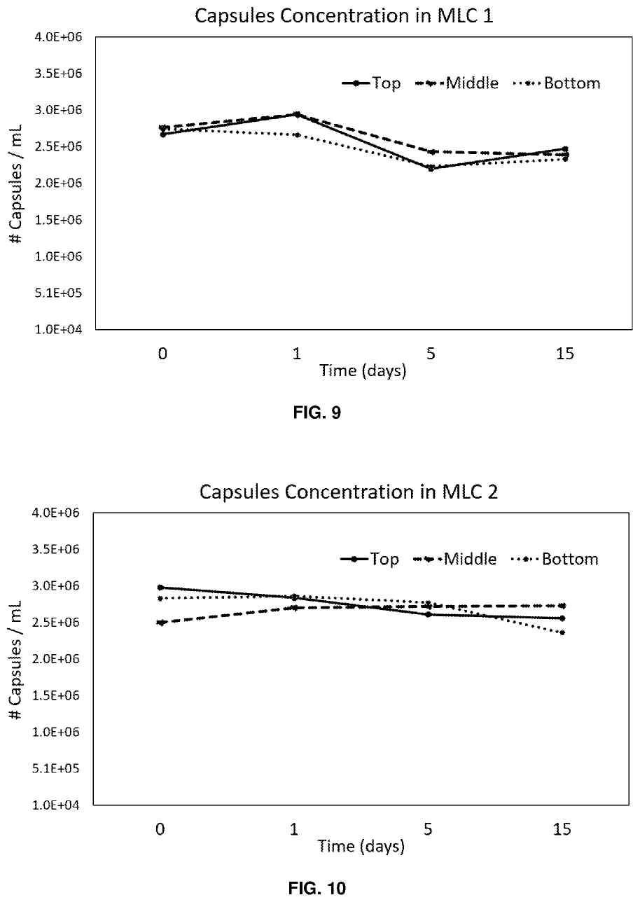

FIGS. 8-10 show the dispersion capacity of the present microstructured liquid composition, having the particulate system as a steady dispersion, even at the 15th day. The use of microfibrillated cellulose at an amount of 0.5% was efficient to provide dispersion for 0.5% wt. of capsules.

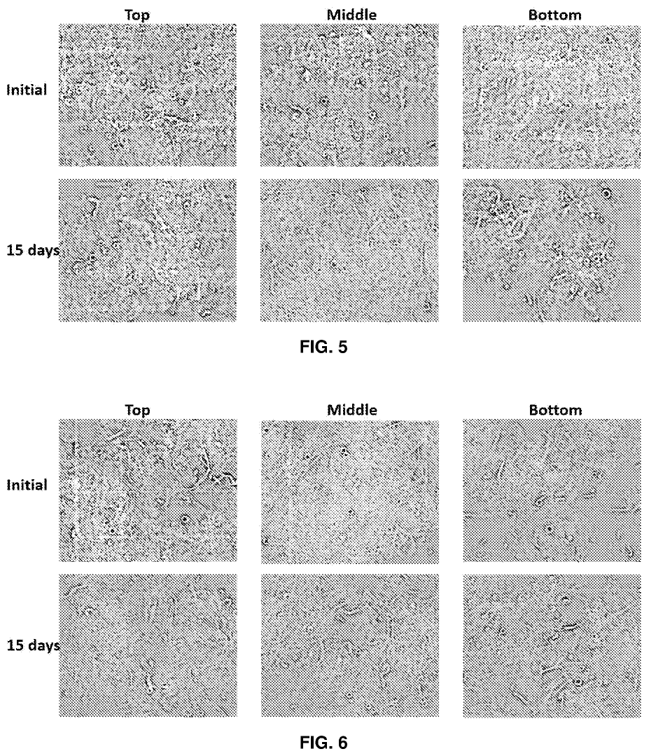

The dispersive capacity of microfibrillated cellulose in particulate systems may be verified in FIGS. 3-6. FIG. 3 is a microscopy of the top, middle and bottom zones of the container of the liquid composition LC, free of microfibrillated cellulose. The microscopy of the initial mixture evidences a homogenously dispersion that degrades in the 15th day.

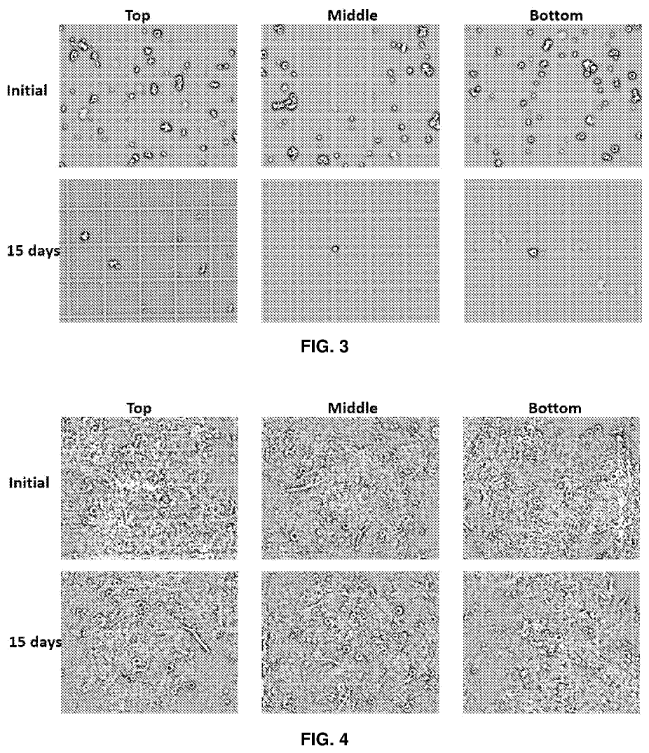

FIGS. 4-6. Are microscopies of the top, middle and bottom zones of the container of the liquid compositions MLC 0.5, MLC 1 and MLC 2 having, respectively, 0.5%. 1% and 2% wt. of microfibrillated cellulose, plus 0.5% of microcapsules. The microscopies of mixtures in the 15th day show no degradation of the homogeneity of the dispersion.

### Example 2

In order to evaluate the MFC ability to stabilize not only particles that tends to float, but also denser particles that tend to deposit on the bottom two experiments were done using the same amount of particles (0.5% wt.):
Suspension 1—reference with no MFC addition—LC
Suspension 2—reference with 1% wt. of MFC—MLC 1

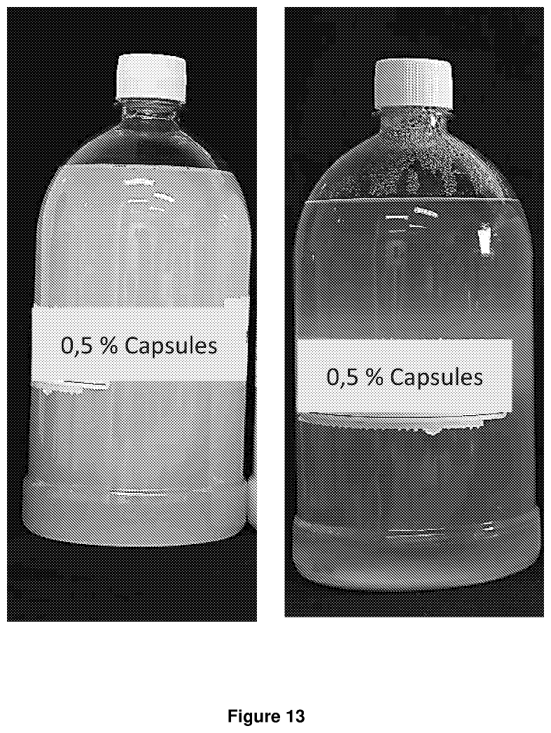

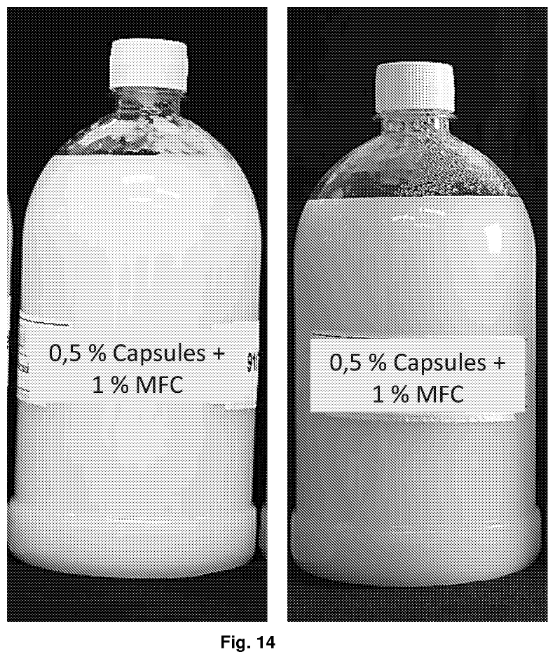

Suspensions 1 and 2 stabilities were evaluated over 15 days where samples from three different levels were taken and the number of particles was counted (FIGS. 13 and 14).

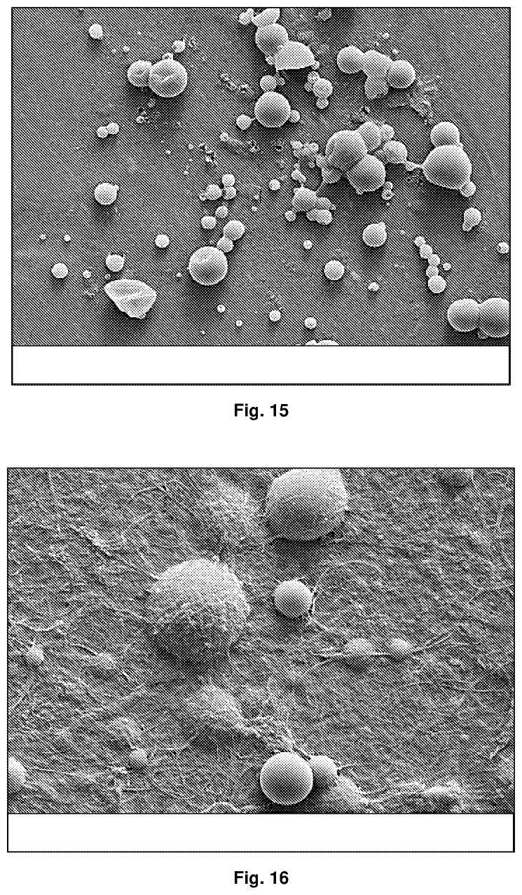

Scanning electron microscopy was also used in order to verify the “encapsulation” of the particles by the MFC (FIGS. 15 and 16).

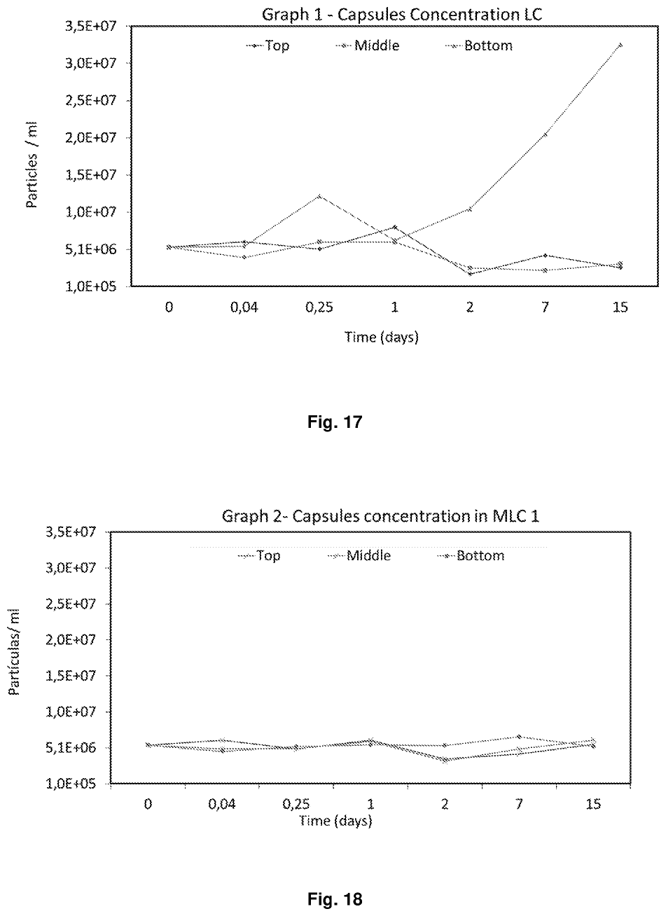

In FIGS. 17 and 18, the graphs represent the number of particles counted at three different suspension levels: top, middle and bottom. Such measurements were carried out with the support a microscope and the results in the graph of FIG. 17 (LC) shows the “movement” of the suspended particles towards the bottom of the flask. In other words, the curves in this graph show the instability of the suspension which is an undesired phenomenon. These results confirm the observation done in SEM images (FIGS. 15 and 16).

Contrarily, when 1% wt. of MFC was added to the suspension as per the graph in FIG. 18 (MLC 1), particles were stabilized across suspension even after 15 days, i.e., particles do not migrate to the bottom of the flask. That shows the MFC ability in suppressing the particles to deposit on the bottom (caking, sedimentation).

### Example 3ç

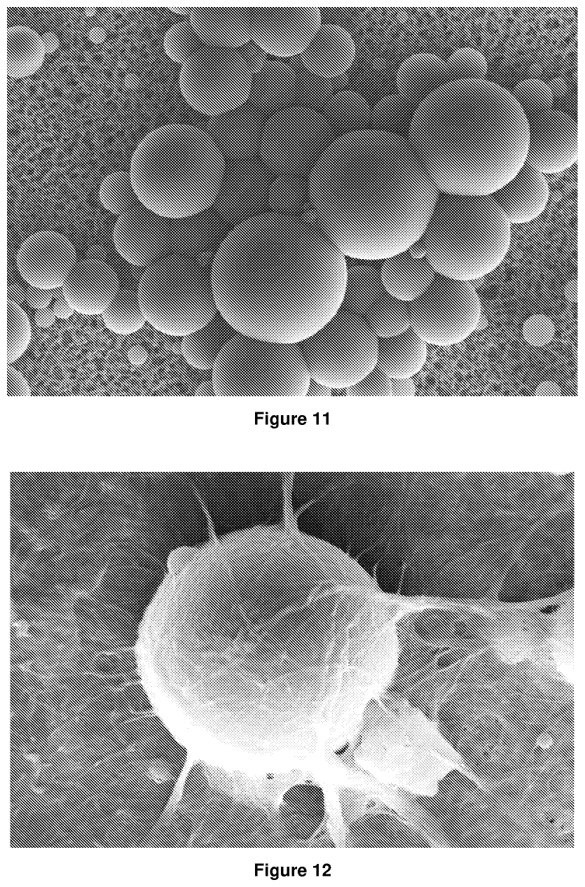

Further, the FIGS. 4-6 also show the network structure of microfibrillated cellulose in a stabilized liquid composition comprising a particulate system, which can be visualized at FIG. 12. The network provides structuring to the particulate system and maintains a local microdispersion, as well as a local stability for the particulate system. For comparison, FIG. 11 is the capsules in the liquid composition LC free of microfibrillated cellulose and freely moving through the liquid medium.

Further, from FIG. 12, it is noticed the protective feature of the microfibrillated cellulose to the particulate system elementary components. The microfibrillated cellulose, when used in the amounts herein disclosed, provides protection to the particulate system elementary components from shearing forces and agitation impact while, at the same time, good mixing characteristics to the liquid composition.

In this sense, another object of the invention is to provide the use of a microfibrillated cellulose as a protective agent for a particulate system in a liquid composition. A protective agent is an agent that is capable of providing physical protection to a particulate system elementary component, such as particles or capsules, from shearing forces or impacts. The physical resistance is provided against, for instance, impacts from the agitator or physical abrasion from the agitator or the medium under agitation.

A liquid composition comprising a particulate system is usually prepared by mixing the particulate system and the diluent. Mixing operations usually occur under high shear and/or agitation. Usually an agitator is used to provide the high shear or agitation, caused by the motion of agitator and turbulence of the liquid medium surrounding the agitator. The movement of the agitator at high speeds agitates the liquid medium, and the mixing composed of the diluent and particulate system. However, under high speeds and shear, the agitator causes the particulate system to breakdown, erode, and leads to deleterious effects in the elementary components of the particulate system to the liquid medium, even causing losses and unbalances in the final product perceived amounts. The use of a microfibrillated cellulose as a protective agent for a particulate system in a liquid composition provides protection for the particulate system elementary components against the impacts of the agitator, preserving the particles or capsules integrity and avoiding early breakdown events that leads to processing costs and incorrect active contents due to the loss of particles or capsules contents to the medium. The use of the microfibrillated cellulose as a protective agent for the particulate system allows the application of moderate/higher shear forces of agitation in the liquid composition, shortening the mixing times or even providing protection to fragile particulate systems when the diluents or the process demand longer mixing times.

In this sense, the microfibrillated cellulose is also a protective agent to abrasion caused by the agitator or by the turbulent liquid medium. Agitation in the liquid medium causes turbulent flow of the liquid that leads to shearing forces between the particulate system elementary components, the surrounding liquid, or both, which may also be another source of wear of the particulate system elementary components. The microfibrillated cellulose acts as a protective agent against the abrasion and wear of the particulate system elementary components, either particles or capsules, or a mixture of both, preserving and avoiding undue losses. To achieve such compositions, the present invention provides the use of a microfibrillated cellulose as an impact protective agent in the manufacture of a liquid composition comprising a particulate system, preferably from 0.5% to 5.0% of a microfibrillated cellulose in weight of the total composition, more preferably 1.0%.

1.-9. (canceled)

10. A liquid suspension comprising: a liquid, a particulate system of particles suspended in the liquid, and a suspension stabilizer agent comprising a microfibrillated cellulose.

11. The liquid suspension of claim 10, wherein the amount of the suspension stabilizer agent in the liquid suspension is from 0.5% to 5.0%, based on the total weight of the suspension.

12. The liquid suspension of claim 10, wherein the amount of the suspension stabilizer agent in the liquid suspension is about 1.0%, based on the total weight of the suspension.

13. The liquid suspension of claim 10, wherein the particles have diameters from about 0.2 to 2 μm.

14. The liquid suspension of claim 10, wherein the particles have diameters of about 0.2 to 1 μm.

15. The liquid suspension of claim 10, wherein the particles have diameters of about 0.6 μm.

16. The liquid suspension of claim 10, wherein the a microfibrillated cellulose forms a structured network in the liquid suspension.

17. The liquid suspension of claim 10, wherein the particles are selected from the group consisting of powder, microfiber, microparticles, microspheres, and mixtures thereof.

18. The liquid suspension of claim 10, wherein the particles comprise a releasing element selected from the group consisting of a perfume, flavorant, softening agent, fabric conditioning agent, a vitamin, an organic acid, a pharmaceutical, a cosmetic active, antistatic agent, anti-hair loss agent, water repellant agent, a non-stick agent, a skin cooling agent, an antimicrobial agent, a disinfectant agent, an anti-wrinkle agent, a repellant agent, a UV protection agent, a skin conditioning agent, a skin nutrition agent, and mixtures thereof.

19. The liquid suspension of claim 10, wherein the liquid comprises water, ethanol, or a mixture thereof.

20. The liquid suspension of claim 10, wherein the liquid suspension further comprises a surfactant.

21. A protective agent for a liquid suspension comprising a microfibrillated cellulose as the protective agent, and wherein the microfibrillated cellulose is mixed in a liquid comprising a particulate system.

22. A method of preparing a liquid suspension comprising the steps of:
providing a liquid diluent;
adding particles to the liquid diluent to form a particulate system; and
adding a microfibrillated cellulose to the liquid diluent to form the liquid suspension.

23. The method of claim 22, further comprising the step of mixing the liquid diluent, particles, and the microfibrillated cellulose.

24. The method of claim 22, wherein the liquid diluent and particles are mixed prior to the step of adding the microfibrillated cellulose.

25. The method of claim 22, wherein the amount of the microfibrillated cellulose in the liquid suspension is from 0.5% to 5.0%, based on the total weight of the suspension.

26. The method of claim 22, wherein the amount of the microfibrillated cellulose in the liquid suspension is 1%, based on the total weight of the suspension.

27. The method of claim 22, wherein the particles have diameters from about 0.2 to 2 μm.

28. The method of claim 22, wherein the particles have diameters of about 0.2 to 1 μm.

29. The method of claim 22, wherein the particles are selected from the group consisting of powder, microfiber, microparticles, microspheres, and mixtures thereof.
